(19) United States
(12) Patent Application Publication
(10) Pub. No.: US 2004/0235991 A1
(43) Pub. Date: Nov. 25, 2004

(54) Use of aminouracils for stabilising antistatic organic plastics containing halogen

(76) Inventors: Peter Daute, Beverstedt (DE); Peter Wedl, Bremerhaven (DE); Joerg-Dieter Klamann, Alderley, QLD (AU); Ernst-Udo Brand, Bremerhaven (DE)

Correspondence Address: COGNIS CORPORATION, PATENT DEPARTMENT, 300 BROOKSIDE AVENUE, AMBLER, PA, 19002, US

(21) Appl. No.: 10/482,889

(22) PCT Filed: Jun. 27, 2002
(86) PCT No.: PCT/EP02/07105

(30) Foreign Application Priority Data
Jun. 7, 2001 (DE) 101 32 836.2

(51) Int. Cl. C08K 5/34; C07D 237/00

(52) U.S. Cl. 524/100; 544/224

(57) ABSTRACT

Methods of stabilizing antistatically-finished, halogen-containing organic plastics are described, wherein the methods comprise: (a) providing an aminouracil of the general formula (I):

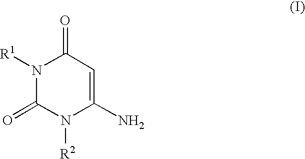

wherein R¹ and R² each independently represent a hydrogen atom, a C₁₋₁₈ alkyl group which can be branched or unbranched and linear or cyclic, or a C₆₋₁₈ aryl group which can be substituted by one or more C₁₋₆ alkyl groups; and (b) combining the aminouracil and the antistatically-finished, halogen-containing organic plastic. Stabilizer compositions containing an aminouracil and PVC formulations containing such stabilizers are also described.

### FIELD OF THE INVENTION

This invention relates to the use of aminouracils for stabilizing antistatically finished halogen-containing organic plastics.

### PRIOR ART

It is known that halogen-containing plastics or molding compositions produced from them tend to undergo degradation or decomposition reactions on exposure to heat stress or on contact with high-energy radiation, for example ultraviolet light.

The stabilizing of PVC during processing has generally involved the use of metal-containing stabilizers based on Pb, Ba, Cd, Sn, Ca and Zn. As long ago as 1940, urea derivatives, such as diphenylthiourea for example, were proposed for stabilizing PVC (cf. Gächter/Müller, “Kunststoff-Additive”, Carl Hanser Verlag 1989, p. 312). These compounds are generally used in combination with metal-containing stabilizers because the long-term stability they provide on their own is mostly inadequate.

It is also known that plastics including PVC are pronounced nonconductors, i.e. are excellent electrical insulators. The effect of this property is that the surface becomes electrically charged and molded parts soon become soiled and unsightly in appearance because of the dust they therefore attract. These unfavorable properties can largely be prevented by antistatic agents (antistatics).

Antistatics are divided into external and internal antistatics. External antistatics are products which are applied as a thin layer to the surface of PVC moldings. The disadvantage of this surface coating lies in the poor durability of the antistatic effect so that the protective effect gradually diminishes and an aftertreatment has to be applied, above all after rinsing and washing. Internal antistatics are part of the PVC compound and are incorporated in the PVC together with other additives. The major advantage of internal antistatics is the permanence of their effect.

Solutions of quaternary ammonium salts, amine derivatives and special phosphoric acid esters are used as external antistatic agents. Hygroscopic substances, such as glycerol, glycol and other polyols, are also suitable. Whereas external antistatics are not problematical to apply, several criteria have to be satisfied in the use of internal antistatics. Thus, it is known to the expert that internal antistatics seriously impair the thermal stability of PVC compounds, the most adverse effect being exhibited by quaternary ammonium compounds which are particularly effective antistatics. Accordingly, these products are not suitable as internal antistatics for PVC, particularly rigid PVC (cf. Kunststoff-Handbuch, Vol. 2/1: Polyvinylchlorid, Hans. K. Felger (Ed.), Munchen 1985, page 730). The same applies to amine derivatives, for example ethoxylated fatty amines.

### DESCRIPTION OF THE INVENTION

The problem addressed by the present invention was to provide substances which would be suitable for stabilizing antistatically finished halogen-containing organic plastics, more particularly antistatically finished PVC, against thermal and/or photochemical degradation. In particular, these substances would be capable of ensuring the thermal stability of PVC finished with internal antistatics, more particularly quaternary ammonium compounds and amine derivatives.

The present invention relates to the use of aminouracils for stabilizing antistatically finished halogen-containing organic plastics against thermal and/or photochemical degradation.

In one embodiment, the compounds (I) are used to stabilize antistatically finished PVC against thermal and/or photochemical degradation. The stabilization of PVC finished with internal antistatics, more particularly quaternary ammonium compounds and amine derivatives, is particularly preferred.

Aminouracils (I) correspond to formula (I):

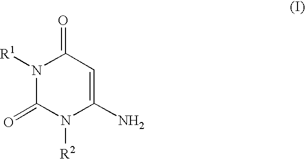

in which R¹ and R² independently of one another represent hydrogen or an unbranched or branched, linear or cyclic alkyl group containing 1 to 18 carbon atoms or an aryl group containing 6 to 18 carbon atoms which may optionally be substituted by one or more alkyl groups each containing 1 to 6 carbon atoms. Dimethylaminouracil (I*) is most particularly preferred for the purposes of the invention.

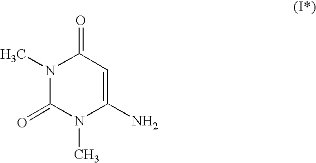

The present invention also relates to stabilizer compositions for stabilizing antistatically finished halogen-containing organic plastics, more especially PVC, against thermal and/or photochemical degradation, characterized in that these compositions contain one or more aminouracils (I). In one embodiment, the stabilizer compositions contain one or more perchlorates besides the compounds (I).

Perchlorates in the context of the present invention are metal salts and ammonium salts of perchloric acid. Examples of perchlorates suitable for the purposes of the invention are those corresponding to the formula M(CIO₄)ₙ, where M stands in particular for ammonium, Li, Na, K, Mg, Ca, Sr, Zn, Al, La or Ce. The index n has a value of 1, 2 or 3 according to the valency of the cation M. The perchlorate salts may be complexed with or dissolved in alcohols, for example polyols, cyclodextrins or ether or ester alcohols. Ester alcohols also include polyol partial esters. In the case of polyhydric alcohols or polyols, dimers, trimers, oligomers and polymers thereof—such as di-, tri-, tetra and polyglycols and di-, tri- and tetrapentaerythritol or polyvinyl alcohols in various degrees of polymerization—may also be used. Perchlorate/alcohol complexes specifically include the types known to the expert from EP-B-394 547, page 3, lines 37 to 56.

The perchlorate salts may be used in the form of various standard preparations, for example as salts or solutions in water or organic solvents either as such or applied to a carrier material, such as PVC, Ca silicate, zeolites or hydrotalcites or “bound” by chemical reaction into a hydrotalcite or another layer lattice compound. Preferred polyol partial ethers are glycerol monoethers and glycerol monothioethers.

The perchlorates may be used either individually or in the form of mixtures with one another.

The present invention also relates to a process for stabilizing antistatically finished halogen-containing organic plastics, more especially PVC, against thermal and/or photochemical degradation, characterized in that one or more aminouracils (I) is/are added to the plastics which contain in particular internal antistatic agents. In a preferred embodiment, the components, i.e. the antistatically finished PVC and the compounds (I), are thoroughly mixed in suitable units.

The stabilizer compositions according to the invention may advantageously be incorporated by the following methods:

as an emulsion or dispersion (for example in the form of a paste-form mixture, in which case an advantage of the combination according to the invention is the stability of the paste);

as a dry blend during the mixing of added components or polymer mixtures;

by direct introduction into the processing unit (for example calendar, mixer, kneader, extruder and the like) or

as a solution or melt.

The present invention relates to a stabilized PVC which contains on the one hand one or more antistatic agents, more particularly internal antistatic agents, and on the other hand one or more compounds (I). A stabilized and antistatically finished PVC such as this may be produced in known manner, for which purpose the compounds (I) or a stabilizer combination according to the invention and antistatic agents and optionally other typical additives for plastics are mixed with PVC in units known per se, such as the processing units mentioned above. In a preferred embodiment, the PVC additionally contains one or more perchlorates.

The stabilized antistatic-containing PVC preferably contains the compounds (I) in a quantity of 0.01 to 2.0 phr and more particularly 0.01 to 0.5 phr. The expression “parts per hundred resin” (phr) familiar to the expert indicates how many parts by weight of the component are present in the PVC, based on 100 parts by weight PVC.

The stabilized antistatic-containing PVC preferably contains the perchlorates in a quantity of 0.01 to 2.0 phr and more particularly 0.01 to 0.5 phr.

The PVC stabilized in accordance with the invention may be brought into the required shape by known methods such as, for example, calendering, extrusion, injection molding, sintering or spinning, extrusion blowing or processing by the plastisol process.

Plastisol processing, extrusion and calendering are particularly preferred processes for processing the PVC stabilized in accordance with the invention.

The PVC stabilized in accordance with the invention is suitable for rigid, semirigid and flexible formulations.

Halogen-Containing Organic Plastics

The antistatically finished halogen-containing organic plastics to be stabilized with the compounds (I) or with the compositions according to the invention are, in particular, chlorine-containing polymers or recyclates thereof. Examples of such chlorine-containing polymers or recyclates to be stabilized are polymers of vinyl chloride, vinyl resins containing vinyl chloride units in their structure, such as copolymers of vinyl chloride and vinyl esters of aliphatic acids, more particularly vinyl acetate, copolymers of vinyl chloride with esters of acrylic and methacrylic acid and with acrylonitrile, copolymers of vinyl chloride with diene compounds and unsaturated dicarboxylic acids or anhydrides thereof, such as copolymers of vinyl chloride with diethyl maleate, diethyl fumarate or maleic anhydride, post-chlorinated polymers and copolymers of vinyl chloride, copolymers of vinyl chloride and vinylidene chloride with unsaturated aldehydes, ketones and others, such as acrolein, crotonaldehyde, vinylmethyl ketone, vinylmethyl ether, vinylisobutyl ether and the like; polymers of vinylidene chloride and copolymers thereof with vinyl chloride and other polymerizable compounds; polymers of vinyl chloroacetate and dichlorodivinyl ether; chlorinated polymers of vinyl acetate, chlorinated polymeric esters of acrylic acid and α-substituted acrylic acid; polymers of chlorinated styrenes, for example dichlorostyrene; chlorinated polymers of ethylene; polymers and post-chlorinated polymers of chlorobutadiene and copolymers thereof with vinyl chloride; and mixtures of the polymers mentioned with one another or with other polymerizable compounds.

Graft polymers of PVC with EVA, ABS and MBS are also included. Other preferred substrates are mixtures of the above-mentioned homo- and copolymers, more particularly vinyl chloride homopolymers, with other thermoplastic and/or elastomeric polymers, more particularly blends with ABS, MBS, NBR, SAN, EVA, CPE, MBAS, MA, PMMA, EPDM and polylactones.

Suspension and bulk polymers and emulsion polymers are also preferred.

The particularly preferred chlorine-containing polymer is polyvinyl chloride, more especially suspension polymer and bulk polymer.

In the context of the invention, PVC is also understood to include copolymers or graft polymers of PVC with polymerizable compounds, such as acrylonitrile, vinyl acetate or ABS, in the form of suspension, bulk or emulsion polymers. PVC homopolymer—even in combination with polyacrylates—is preferred.

Recyclates of chlorine-containing polymers are also suitable, recyclates being the polymers described in detail in the foregoing which have been damaged by processing, use or storage. PVC recyclate is particularly preferred. The recyclates may also contain small quantities of foreign materials such as, for example, paper, pigments, adhesives, which are often difficult to remove. These foreign materials may even emanate from contact with various substances during use or working up, including for example fuel residues, paint/lacquer, metal traces and initiator residues.

### EXAMPLES

Substances Used

Norvinyl S 6260 = S-PVC (K value = 62) (Hydro Polymers AB)
Vestolith E 6007 = E-PVC (K value = 60) (Degussa)
Vestinol AH = dioctyl phthalate (BASF)
Kronoss 2220 = titanium dioxide (Kronos Titan)
Edenol D 81 = epoxidized soya oil (Cognis, Germany)
Zeolite A (Degussa)
Zinc laurate
Calcium stearate
PEG 200 monolaurate (Cognis, Germany)
Dehydat 80 X = antistatic: paraffin sulfonate, Na salt (Cognis, Germany)
Na perchlorate = 50% by weight solution of Na perchlorate in water
1,3-Dimethylaminouracil (commercial product of Knoll)

### Examples 1 to 8

Table 1 below shows on the one hand the individual ingredients of the test formulations and, on the other hand, the test results obtained. The numbers of the Examples are shown in the first line of the Table. The quantities of the individual components are expressed in phr (phr=parts per hundred resin) which indicates how many parts by weight of the particular component are present in the PVC (based on 100 parts by weight PVC) after addition of the composition. Accordingly, the formulations each contain 100 parts PVC (sum of Norvinyl S 6260 and Vestolith E 6007).

Examples 4 and 5, 7 and 8 correspond to the invention. Examples 1, 2, 3 and 6 are intended for comparison.

The test formulations were subjected—partly or completely—to the following measurements:

Stability test under heat stress. Strips were produced from the formulations and tested for static thermal stability at 170° C. The strips were produced by homogenizing and plasticizing the PVC powder mixture and the formulation components mentioned for 5 minutes at 170° C. on a laboratory roll mill. Test specimens measuring 17×17 mm were cut out from the ca. 0.5 mm thick strips thus produced. The test specimens were placed in a heating cabinet at 170° C. on glass plates on rotating trays and removed at 15-minute intervals until all the test specimens were “burnt” (i.e. were black in color).

Color measurement on strips. In addition, the L*,a*,b*-method (cf. DIN 6174) known to the expert was applied to the test strips used for further characterization. The b values determined are shown in Table 1. A commercially available instrument (Dr. Lange “Micro Color”) was used for the measurements.

TABLE 1

| | E1 | E2 | E3 | E4 | E5 | E6 | E7 | E8 |
|---|---|---|---|---|---|---|---|---|
| Formulations - ingredients in | | | | | | | | |
| phr | | | | | | | | |
| Norvinyl S 6260 | 75 | 75 | 75 | 75 | 75 | 75 | 75 | 75 |
| Vestolith E 6007 | 25 | 25 | 25 | 25 | 25 | 25 | 25 | 25 |
| Vestinol AH | 30 | 30 | 30 | 30 | 30 | 30 | 30 | 30 |
| Kronoss 2220 | 2 | 2 | 2 | 2 | 2 | 2 | 2 | 2 |
| Edenol D 81 | 3 | 3 | 3 | 3 | 3 | 3 | 3 | 3 |
| Zeolite A | 0.27 | 0.27 | 0.27 | 0.27 | 0.27 | 0.27 | 0.27 | 0.27 |
| Zinc laurate | 0.2 | 0.2 | 0.2 | 0.2 | 0.2 | 0.2 | 0.2 | 0.2 |
| Calcium stearate | 0.15 | 0.15 | 0.15 | 0.15 | 0.15 | 0.15 | 0.15 | 0.15 |
| PEG 200 monolaurate | | | 10 | 10 | 10 | | | |
| Dehydat 80 X | | | | | | 3 | 3 | 3 |
| Na perchlorate* | | | | | 0.2 | | | 0.2 |
| 1,3-Dimethylaminouracil | | 0.28 | | 0.28 | 0.28 | | 0.28 | 0.28 |
| Color measurement on strips - | | | | | | | | |
| lightness\[b] | | | | | | | | |
| Without storage | 5.1 | 3.7 | 4.5 | 4.6 | 4.1 | 5.6 | 3.4 | 3.4 |
| 15 mins. at T = 170° C. | 12.2 | 6.1 | 15.4 | 7.0 | 7.0 | 13.4 | 7.3 | 5.6 |
| 30 mins. at T = 170° C. | 18.1 | 7.1 | | 10.6 | 11.4 | | 9.9 | 7.4 |
| End of stability after storage | | | | | | | | |
| in an oven (T = 170° C.) | | | | | | | | |
| [mins.] | 75 | 90 | 30 | 60 | 75 | 45 | 75 | 90 |

*Na perchlorate: 50% solution in water

1-10 (Canceled)

11. A method of stabilizing an antistatically-finished, halogen-containing organic plastic, said method comprising:
(a) providing an aminouracil of the general formula (I): 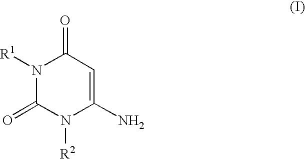
wherein R¹ and R² each independently represent a hydrogen atom, a c₁₋₁₈ alkyl group which can be branched or unbranched and linear or cyclic, or a C₆₋₁₈ aryl group which can be substituted by one or more C₁₋₆ alkyl groups; and
(b) combining the aminouracil and the antistatically-finished, halogen-containing organic plastic.

12. The method according to claim 11, wherein the aminouracil comprises dimethylaminouracil according to formula (I*):

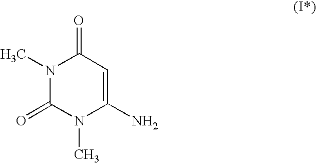

13. The method according to claim 11, wherein the antistatically-finished, halogen-containing organic plastic comprises a polyvinyl chloride formulation.

14. The method according to claim 12, wherein the antistatically-finished, halogen-containing organic plastic comprises a polyvinyl chloride formulation.

15. The method according to claim 11, further comprising combining one or more perchlorates with the aminouracil and the antistatically-finished, halogen-containing organic plastic.

16. The method according to claim 11, wherein the aminouracil is combined with the antistatically-finished, halogen-containing organic plastic in an amount of from 0.01 to 2.0 phr.

17. A composition for stabilizing an antistatically-finished, halogen-containing organic plastic, said composition comprising one or more aminouracils of the general formula (I): 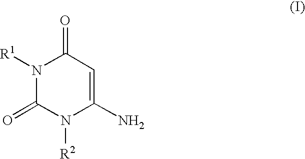
wherein R¹ and R² each independently represent a hydrogen atom, a C₁₋₁₈ alkyl group which can be branched or unbranched and linear or cyclic, or a C₆₋₁₈ aryl group which can be substituted by one or more C₁₋₆ alkyl groups.

18. The composition according to claim 17, wherein the one or more aminouracils comprises dimethylaminouracil.

19. The composition according to claim 17, further comprising one or more perchlorates.

20. A stabilized PVC formulation comprising one or more antistatic agents and one or more aminouracils of the general formula (I): 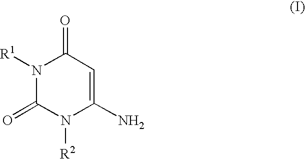
wherein R₁ and R₂ each independently represent a hydrogen atom, a C₁₋₁₈ alkyl group which can be branched or unbranched and linear or cyclic, or a C₆₋₁₈ aryl group which can be substituted by one or more C₁₋₆ alkyl groups.

21. The stabilized PVC formulation according to claim 20, wherein the one or more aminouracils comprises dimethylaminouracil.

22. The stabilized PVC formulation according to claim 20, wherein the one or more antistatic agents comprises an internal antistatic agent.

23. The stabilized PVC formulation according to claim 21, wherein the one or more antistatic agents comprises an internal antistatic agent.

24. The stabilized PVC formulation according to claim 20, further comprising one or more perchlorates.

25. The stabilized PVC formulation according to claim 21, further comprising one or more perchlorates.

26. The stabilized PVC formulation according to claim 23, further comprising one or more perchlorates.
